(19) United States
(12) Patent Application Publication
(10) Pub. No.: US 2018/0299424 A1
(43) Pub. Date: Oct. 18, 2018

(54) SYSTEM AND METHOD FOR NUCLEOTIDE SEQUENCING

(71) Applicants: Bharath Takulapalli, Chandler, AZ (US); Stuart Lindsay, Phoenix, AZ (US)

(72) Inventors: Bharath Takulapalli, Chandler, AZ (US); Stuart Lindsay, Phoenix, AZ (US)

(21) Appl. No.: 15/524,147

(22) PCT Filed: Nov. 10, 2015
(86) PCT No.: PCT/US2015/059812

§ 371 Date: May 3, 2017

Related U.S. Application Data
(60) Provisional application No. 62/078,730, filed on Nov. 12, 2014.

(51) Int. Cl. G01N 33/487; G01N 27/414

(52) U.S. Cl. CPC G01N 33/48721; G01N 27/4146; G01N 27/4145

(57) ABSTRACT

The present invention provides a system and method of sequencing complex molecules including DNA, RNA, proteins, and glycans. The method includes the steps of modifying a field effect nanopore transistor device with chemical recognition molecules, translocating the complex molecule into the field effect nanopore transistor device, applying bias potential to the silicon gate of the field effect nanopore transistor device, and measuring the resulting change in drain current across the source drain contacts.

### CROSS-REFERENCE TO RELATED APPLICATIONS

This application claims the benefit of U.S. Provisional Patent Application No. 62/078,730, filed Nov. 12, 2014, which is incorporated herein by reference as if set forth in its entirety.

### STATEMENT REGARDING FEDERALLY SPONSORED RESEARCH

This invention was made with government support under R21 HG006314 awarded by The National Institutes of Health. The government has certain rights in the invention.

### BACKGROUND OF THE INVENTION

The disclosure relates, in general, to the analysis of nucleic acids and, more particularly, to a system and method for nucleotide sequencing based on leveraging a field effect transitor nanopore device.

Whole genome sequencing offers the ability to understand the genome and its function. For example, genome sequencing can lead to the development of effective medicines. In general, current whole genome sequencing technologies can be expensive, slow, and incur significant error rates as related to the calling of base pairs (bp) in the nucleotide sequence. While the cost of whole genome sequencing has been reduced from $1 billion for the Human Genome Project a decade ago to approximately $1,000 per genome as of 2012, it would be useful to further lower the per genome cost of sequencing.

With respect to error frequency and read-length, the quality of genome sequencing data is often determined using Phred base calling, a computer program for identifying a base sequence, and a calculated Phred quality score (q), which is assigned to each base. The higher the score, the more accurate the base call. It has been observed that the shorter the base read-length (i.e., the length of a DNA or RNA sequence in nucleotide bases or bp), the higher the sequence coverage required for high quality sequencing. The longer the base read length, the less need there is for sequence coverage to obtain quality sequencing.

Various sequencing methods have been used in the past. The Sanger method was used in the Human Genome Project. There, the genome was sequenced six times (sequence coverage of six), the base read-length was 500-600 bp with the Sanger method, and the Project is estimated to be 99.99% accurate. Current massively parallel sequencing technologies use the shotgun sequencing method, along with first genome-code as a reference, to align the data to achieve (consensus) accuracy of 99.99% (i.e., one error in every 10,000 base calls) with a q-score of 40. One next generation sequencing technology includes 454 sequencing, which has a read length of 300-400 bp and sequences with a 10-fold coverage. By comparison, Illumina dye and SOLiD (Sequencing by Oligonucleotide Ligation and Detection) sequencing methods, which have a read length of 50-100 bp, may require 30-fold sequence coverage to achieve 99.99% accuracy.

Given that there are greater than 3 billion base pairs in the human genome, an accuracy of 99.99% may lead to over 300,000 errors. Accordingly, it is desirable to further increase the accuracy of a genome sequencing technology. Complete Genomics reported full genome sequencing with 99.999% accuracy in 2009, but for this, the depth of sequence coverage required was 90 (i.e., every base had to be sequenced 90 times). In cases of de novo sequencing without a reference genome, the error rates are expected to be higher (raw read accuracy). Indeed, the highest quality reported in de novo sequencing is by Pacific Biosciences with 99.999% accuracy using read lengths of 5 kilobase pairs (Kb) to 10 Kb. By comparison, Oxford Nanopore has reported an error rate of close to 4% with the ultimate goal of reducing this to below one percent.

Second generation sequencing technologies capable of only short read-lengths have proven sufficient for reading small non-human genomes. However, they are not optimal for many clinical applications of human sequencing technology due to difficulty in accurate alignment such as for resolving repetitive sequences, complex regions, heterozygous alleles, sequencing of RNA transcripts, or ribosomal RNA sequencing. Third generation sequencing methods are capable of long read-lengths, but some of the single molecule techniques are reported to have above 10% error rate in single run. Currently, Pacific Biosciences has the longest read lengths possible (5 Kb) with high accuracy of 99.999% at a sequence coverage of 20. Oxford Nanopore is reported to be working towards 10 Kb long read lengths currently and 100 Kb read lengths in the future.

In yet another aspect, current technologies require anywhere from a few days to a few weeks to sequence a whole genome. Moreover, to the inventors' knowledge, there are no technologies currently available that can read unmodified DNA bases when indirect base-calling is applied. Accordingly, there is need for advanced technologies that are capable of reading long bp lengths, require minimal sequence coverage, minimize error rates, reduce sequencing times, read unmodified DNA bases when indirect base-calling is applied, or a combination thereof.

### SUMMARY OF THE INVENTION

The present invention provides, among other things, a method for rapid genome sequencing by field effect transduction of chemical recognition coupling.

In accordance with one aspect of the present disclosure, a method for sequencing complex molecules including DNA, RNA, poly-peptides, proteins, glycans, polysaccharides and other biopolymers. The method comprises the steps of modifying a field effect nanopore transistor (FENT) device with chemical recognition molecules, translocating the complex molecule into the FENT device, applying bias potential to the silicon gate of the field effect transistor nanopore device, and measuring the resulting change in drain current across the source drain contacts.

In some cases, the FENT comprises a silicon-or-insulator wafer substrate; source and drain regions that are n+ doped; a semiconductor channel that is continuous from source to drain regions and which narrows down into a conical point nanopore at the center; a silicon gate that acts as a back/buried-gate; a gate oxide layer that separates the silicon gate from the semiconductor channel; and where the field effect transistor nanopore device is configured to operate in a fully depleted mode or partially depleted mode, such that a sensed chemical moiety and/or DNA base causes a measurable change in channel conductance. The chemical recognition molecule can be placed on the semiconductor channel surface. The chemical recognition molecule can be imidazole. The height of the chemical recognition molecule layer can be within the range of from 3 Å to 200 ÅThe chemical recognition molecule can comprise a unique molecule or a combination of molecules. The chemical recognition molecule can be located at the edge of the nanopore. The chemical recognition molecule can specifically interact with the detected complex molecules. The chemical recognition molecule can be an antibody coating. The chemical recognition molecules can be complementary DNA bases. The multiple layers of chemical or biomolecules can be sequentially attached to the FENT device for detection of the complex molecules. Sequentially attaching recognition molecules or chemical molecules or biomolecules can comprise one or more of: chemical attachment, light directed attachment, electrochemical attachment, electrolysis-aided attachment, e-beam aided attachment, ion-beam aided attachment, and surface curvature aided attachment. Different surface regions of the FENT device can be coated with different chemical probes, biomolecules, or polymers. The FENT device can be operated with silicon channel biased in one or more of inversion, accumulation, volume inversion, depletion, partial depletion, or full depletion. The FENT device can be biased with an alternating current (AC) signal to filter-out noise. The FENT device can be used to count the material passing through the nanopore.

In another aspect, provided herein is a method of selectively coating a field effect transistor nanopore device with thin films and with chemical recognition molecules.

In a further aspect, provided herein is a method of using arrays of field effect transistor nanopore device to acquire genome sequence information. Each field effect transistor nanopore (FENT) device within the array can be made with different exterior coatings. The method can further comprise electronic components configured so as to read out electrical signals, perform computational data analysis, and base identification.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a cross-sectional perspective view of a schematic illustration of an example fully depleted exponentially coupled field effect nanopore transistor (FENT) device;

FIG. 2 is a chemical structure drawing of an imidazole molecule with a linker;

FIGS. 3A-3C are band diagrams showing FDEC potential coupling. FIG. 3A shows a flat band diagram, FIG. 3B shows fully depleted band bending biased in weak inversion and exposed to buffer/ionic solution, and FIG. 3C shows FDEC potential coupling at MHz frequencies with ˜10 μs/base translocation.

FIG. 4 is a schematic representation of recognition tunneling of current that is specific to deoxy-adinine;

FIG. 5 discriminated distribution of currents for each of the four DNA bases.

FIG. 6 is a plot of current as a function of time for recognition tunneling of deoxyadenosine;

FIGS. 7A-7D are a schematic representation of recognition tunneling for each the four DNA bases;

FIG. 8 is a schematic illustration showing a perspective view of an example FDEC FENT device having a silicon thin-film layer with a thickness of about 100 nm, an oxide gate layer with a thickness of about 400 nm, and a silicon substrate base that acts as a buried/back gate;

FIG. 9 is a cross-sectional perspective view of the example device of FIG. 8 as taken through the central nanopore of the device;

FIG. 10 is an enlarged partial cross-section perspective view of the nanopore of the device of FIG. 9 showing DNA translocating therethrough; and

FIG. 11 is an enlarged partial cross-sectional plan view of the nanopore of FIG. 10 showing DNA translocating therethrough.

Like numbers will be used to describe like parts from Figure to Figure throughout the following detailed description.

### DETAILED DESCRIPTION OF THE INVENTION

The present invention is presented in several varying embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention.

In general, one aspect of the present disclosure includes a system and method for high speed sequencing of DNA oligomers, where the single base discrimination by chemical recognition is coupled with high-frequency sensing of field effect nanopore transistor (FENT) device. FET sensors may be operated as high frequency switching devices to detect chemical recognition events occurring at the device surface with very high speeds. Using an FET nanopore device coated with imidazole (or similar) molecules, it is possible to achieve single base recognition, as these interact with DNA bases while they translocate through the FET nanopore, where the chemical interactions can be read by the underlying FET sensor. Accordingly, embodiments of the present disclosure provide a low-cost, rapid, reduced-error, increased-base-read method for rapid genome sequencing using chemical recognition labeling and detection by an FET nanopore device.

Embodiments of a device may reduce the time for completion of whole genome sequencing to a few hours. In one aspect, the device is a high speed reader of unmodified DNA bases for direct genome sequencing.

In some embodiments, the present disclosure provides a system and method for the high speed acquisition of genome sequence information. For example, a system and method may include an FET nanopore device for the detection of single DNA bases via chemical recognition. FET sensors may be coated with imidazole (or similar) molecules, which chemically interact with DNA bases at the FET device surface. These chemical interactions may serve as chemical recognition events that are rapidly detected by FET sensors operated as high frequency switching devices. Furthermore, alternating current (AC) biasing of a FET nanopore device can be used as an aid to filter-out the back ground noise, to achieve high accuracy DNA sequencing

Embodiments of a method for high speed sequencing of DNA oligomers may include single base discrimination of chemical recognition coupled with high-frequency sensing of field effect transistor nanopore device. Field effect transistor sensors may be operated as high frequency switching devices, to detect chemical recognition events occurring at the device surface with very high speeds. Using field effect transistor nanopore device coated with imidazole (or similar) molecules, it is possible to achieve single base recognition, as these interact with DNA bases while they translocate through the FET nanopore, where the chemical interactions can be read by the underlying FET sensor.

In one aspect, a system and method may include single base discrimination using chemical recognition. There has been extensive works on hydrogen-bond based identification of nucleosides using tunneling current, by modifying a scanning tunneling microscope probe using a variety of complementary organic molecules, including complementary DNA base pairs. More recently, chemical recognition of all four DNA bases has been demonstrated using a scanning tunneling microscope (STM), where the probe was modified with a unique organic molecule. Discrimination of all four bases is possible by measuring the tunneling current across imidazole modified STM (gold) probe and a similarly modified gold surface with individual bases sandwiched between them.

In another embodiment, provided herein is a system in which a FET nanopore device is coated with other detectable molecules to detect complementary bio-polymers, biomolecules, biomarkers, ions, chemical probes or molecules, drug molecules, particles, nano-particles, magnetic particles, cells, enzymes, vesicles, polypeptides, RNA, or the like. For example, FET nanopore coated with antibodies can be used to detect with high selectivity complementary antigens passing through the nanopore. Coating FET nanopore with proteins can be used to detect interacting complementary proteins passing through the nanopore device. FET nanopore devices coated with chemical probes or proteins or enzymes can be used to detect drug molecules. FET nanopore device can be used also for counting of translocation events, such as ions passing through a cell membrane. FET nanopore sensor can be combined with lipid-bilayers or with cell-walls to mimic protein nanopores that transmit ions, small molecules, oligomers, or biopolymers.

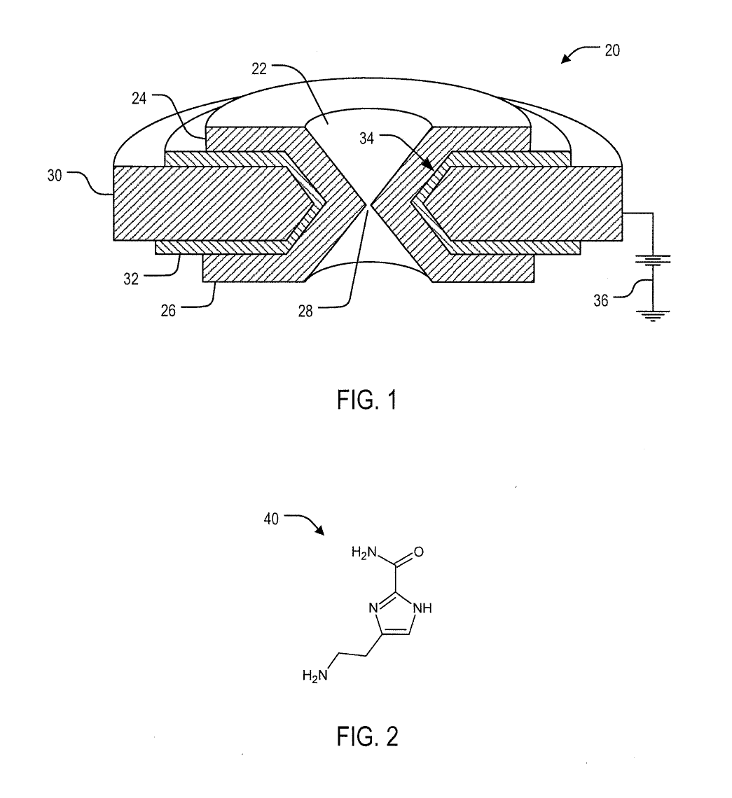

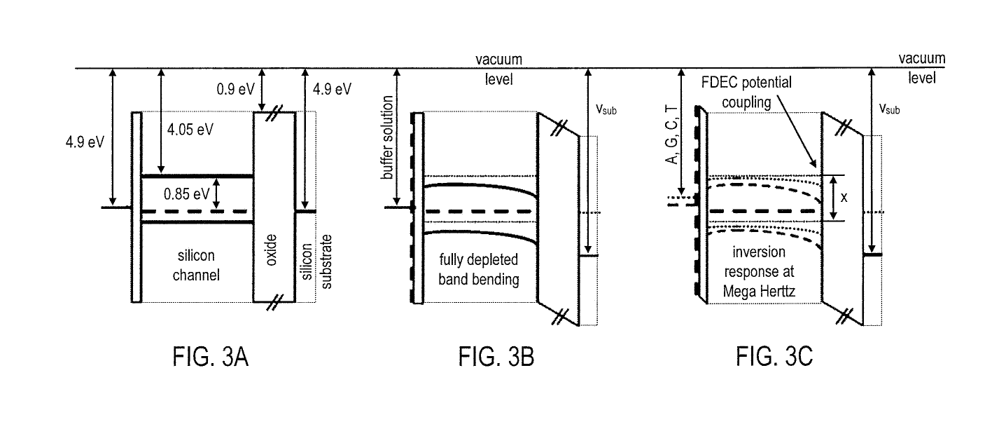

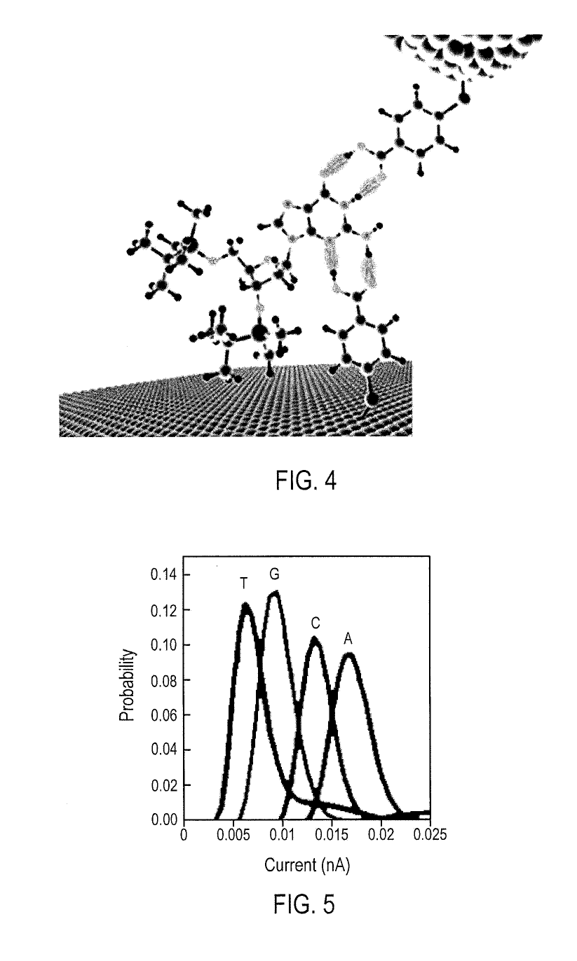

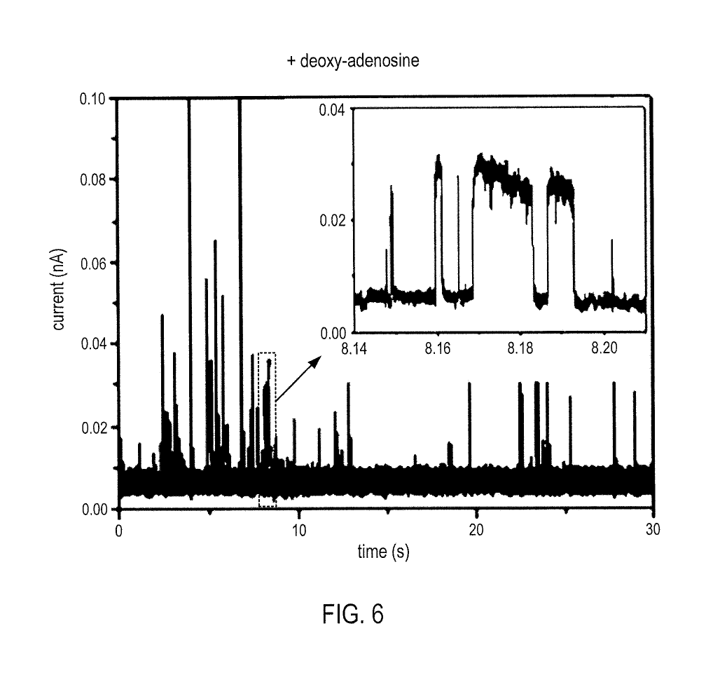

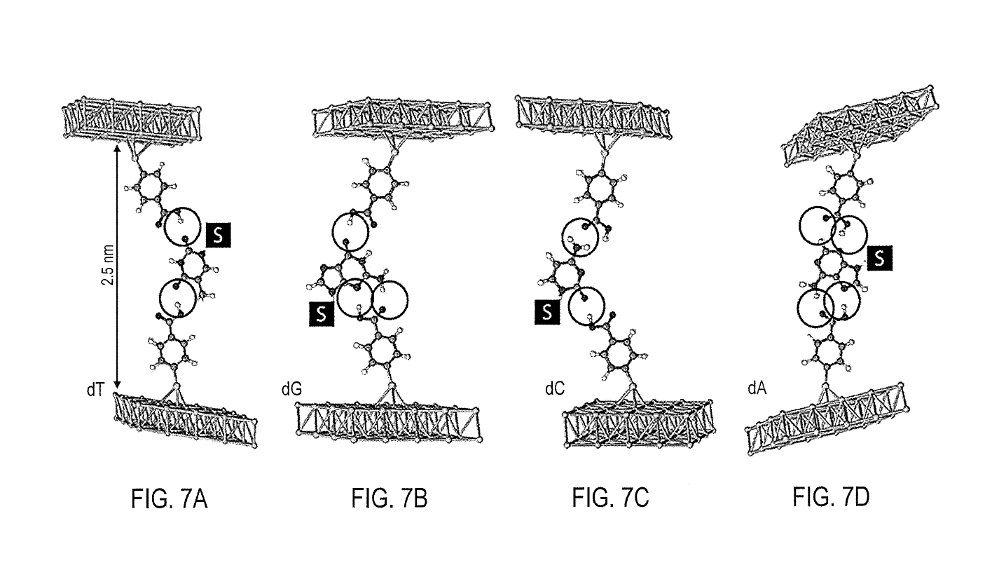

In another aspect, a system and method may include a fully depleted exponentially coupled field effect nanopore device structure. With reference to FIG. 1, a fully depleted exponentially coupled field effect nanopore transistor (FENT) device 20 may be fabricated on silicon-on-insulator wafers using established nano-fabrication techniques. The FDEC FENT device has a semiconductor channel 22 that is continuous from the source region 24 to the drain region 26 and which narrows down into a conical or bi-conical point nanopore 28 at the center. There is a silicon gate 30 that acts as a back/buried-gate, which is separated from the semiconductor channel by a gate oxide layer 32. The source region 24 and drain region 26, which are n+ doped, are connected to external instrumentation via gold bonding pads.

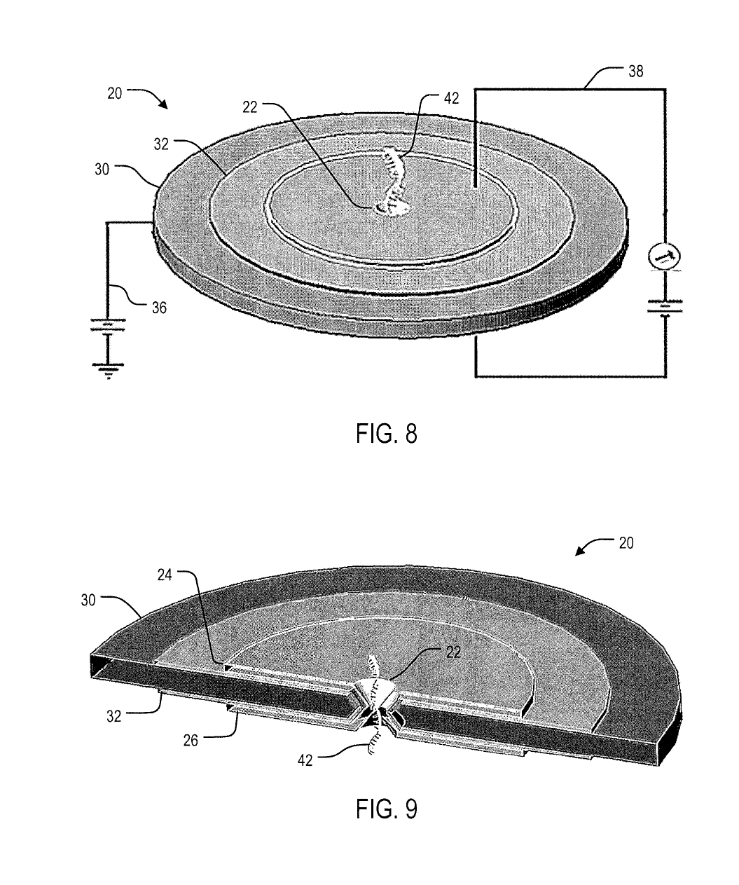

When bias potential is applied to the silicon gate of the FENT device 20, the gate oxide-silicon channel interface 34 is driven into depletion first, followed by full-depletion of the thin film silicon 30, and then into inversion at the gate oxide-silicon channel interface 34. An inversion channel is formed, which is about 20 nm in thickness and continuous along the gate oxide-silicon channel interface 34, from the circular disc of the source region 24 through the conical or bi-conical-point-nanopore 28 to the circular disc of the drain region 26. Drain current is then measured across the source drain contacts (not shown). Device 20 may further include a gate bias 36 and a circuit 38 for source-drain bias and current measurement (see FIG. 8). Alternately FET nanopore device can be operated in accumulation, by forming majority carriers in the channel. In another example, FET nanopore device can be operated in partially depleted mode. And in yet another example, FET nanopore device can be operated in depletion mode. FET nanopore device can also be operated in volume inversion mode where part-of or whole-of the top silicon channel is inverted.

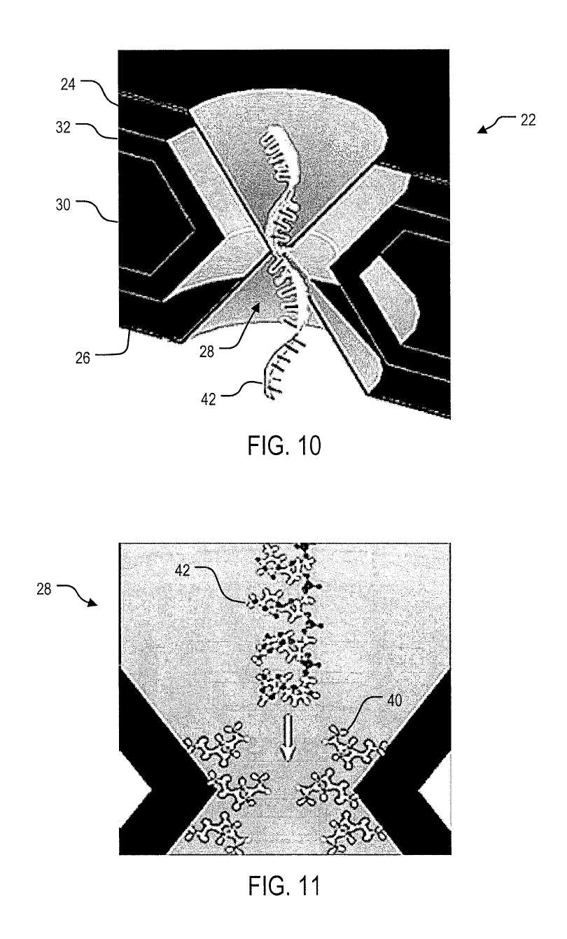

The drain region 26 current measurement is expected to show the similar I-V characteristics as a planar metal oxide semiconductor field effect transistor (MOSFET) device. While current generation commercial MOSFET devices are routinely operated at Giga Hertz switching frequencies, an FENT device may achieve switching speeds up to and above 100 Mega Hertz, as switching speed is inversely proportional to gate oxide thickness. When the FENT device is modified with imidazole or other chemical recognition molecules 40 (see FIG. 2), the measurement of DNA base translocation at above 100,000 events per second can be obtained. Translocation of a DNA oligonucleotide 42 is shown in FIGS. 8-11.

In yet another aspect, a system and method may include FENT devices used as signal transducers for DNA or biomolecule detection. In one aspect, it has been demonstrated that FDEC MOSFET sensors with planar silicon-on-insulator substrates and silicon back-gates can achieve high detection signal transduction. Such sensor technology enables ultra high sensitive detection of chemical and biological species combined with extraordinary selectivity of target molecule detection. FDEC signal transduction is based on the principle that when fully depleted MOSFET devices applied as sensors are operated in inversion regime, any change in charge or potential at the boundary of the fully depleted inverted semiconductor thin-film is internally amplified by the MOSFET capacitive structure, via a variety of coupling mechanisms, yielding orders of magnitude increase in device current response. When biased in full depletion, these devices read, with exponential sensitivity, charge or potential variation at the surface of the device. Alternately FENT nanopore sensor can be operated in partial depletion or volume inversion modes, that also provide high sensitive detection of chemical or biomolecular interactions.

The subject FENT device structure is revolutionary compared to previous FET approaches. The subject FENT sequencer takes advantage of: (1) fully depleted or partially depleted signal transduction; and (2) chemical recognition-coupling using imidazole or other molecules. Specifically, it takes advantage of the specificity of the chemical interaction between imidazole and translocating nucleosides, via fast, instantaneous, transitionary electrostatic bonds (interactions) between the chemical terminations on device surface and translocating DNA bases, at high speeds of translocation. Such interactions occur more readily in aqueous solutions. The specific chemical interaction with individual bases results in efficient potential or charge or work-function coupling with the FENT inversion channel or FENT depletion region or FENT accumulation channel (as the FENT operation case may be), thereby resulting in high signal-to-noise ratio output.

The subject FENT achieves chemical coupling (electrostatic in nature) and corresponding discriminated FENT inversion response by taking advantage of specific imidazole interaction with each of individual DNA bases, while the DNA bases translocate through the nanopore at high speeds. This coupling and corresponding response can be achieved in micro-seconds to milliseconds, which is comparable to high speeds of DNA translocation.

At the nanopore point location, electric field focusing occurs due to conical curvature convoluted around the nanopore center, due to nanopore-edge field amplification. It is verified theoretically and experimentally that electrostatic field varies directly with surface curvature of an object, and ‘electrostatic field extrema along an equipotential contour correspond to curvature extrema.’ And in specific case of 3D conical geometry, field intensity characteristics approach a singularity with not just field intensity extrema, but surface charge, potential, density of state, and surface state interaction extrema occurring at such conical point geometries.

In the subject FENT nanopore structure, electric field focusing is even more extreme due to the nano scale conical surface convoluted around the nanopore center. These amplified fields, states and interactions at the point nanopore location are then Imidazole recognition-coupled to DNA bases and the exponentially transducing FDEC device structure. The response resulting from these double amplification events is read at above Mega Hertz frequencies via source-drain channel current, with very high accuracy.

In one aspect, it may be useful to determine the optimal height of the imidazole layer to functionalize FENT device for chemical recognition. In another embodiment, various methods of functionalizing the FENT device surface or the nanopore surface may be used. These may include, but not limited to, methods such as solution phase coating of recognition molecules or chemical probes on FENT nanopore surfaces, light directed or electron-beam directed or ion-beam directed or chemical motif directed or surface chemistry directed coating of recognition molecules or chemical probes on FENT nanopore surfaces. Electrochemical coating or electrolysis-aided coating of recognition molecules or chemical probes or biochemical or biological molecules can be achieved, on the FENT nanopore surface, by selectively coating specified areas of the devices with specific molecules.

In one embodiment, high speed sequencing of DNA oligomers using single base discrimination by chemically recognition coupled with high frequency sensing of field effector transistor nanopore device is possible. The FET nanopore device is coated with imidazole (or similar) molecules so that discrimination of all four bases by measuring imidazole-DNA interactions requires only a transitory bond or electrostatic interaction between the nucleoside and imidazole rather than the formation of actual bonds. Greater focusing of the electrical field is also possible due to the nanoscale conical surface convoluted around the nanopore center. This response from the amplified electrical field and imidazole coating is read at above Megahertz frequencies via source-drain inversion or accumulation or depletion channel current.

The present invention has been described in terms of one or more preferred embodiments, and it should be appreciated that many equivalents, alternatives, variations, and modifications, aside from those expressly stated, are possible and within the scope of the invention.

Each reference identified in the present application is herein incorporated by reference in its entirety.

While present inventive concepts have been described with reference to particular embodiments, those of ordinary skill in the art will appreciate that various substitutions and/or other alterations may be made to the embodiments without departing from the spirit of present inventive concepts. Accordingly, the foregoing description is meant to be exemplary, and does not limit the scope of present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

### REFERENCES

Takulapalli, Bharath, et al. J. Am. Chem. Soc., 2008, pp. 2226-33, Vol. 130.
Tuchband, Michael, et al. Rev. Scientific Instr., January 2012, p. 015102, Vol. 83.
Takulapalli, Bharath. ACS Nano, 2010, pp. 999-1011, Vol. 4, No. 2.
Liang, Feng, et al. Org. Biomol. Chem., November 2012, pp. 8654-59, Vol. 10, No. 43.
Lindsay, Stuart, et al. Nanotechnology, July 2010, p. 262001, Vol. 21, No. 26.
Liang, Feng, et al. Chemistry, May 2012, pp. 5998-6007, Vol. 18, No. 19.
Huang, Shuo, et al. Nature Nanotechnol., November 2010, Vol. 213.
Fuhrman, Alexander, et al. Biophysical J., May 2012, pp. 2381-90, Vol. 102.
Huang, Shuo, et al. J. Phys. Chem., December 2010, pp. 20443, 48, Vol. 114, No. 48.
Chang, Shuai, et al. Nanotechnol., 2012, p. 425202, Vol. 23.
Chang, Shuai, et al. J. Am. Chem. Soc., September 2011, pp. 14267-69, Vol. 133, No. 36.

We claim:

1. A method of sequencing complex molecules, selected from the group consisting of DNA, RNA, proteins, and glycans, the method comprising the steps of:
(a) modifying a field effect nanopore transistor (FENT) device with chemical recognition molecules;
(b) translocating said complex molecule into the field effect transistor nanopore device;
(c) applying bias potential to the silicon gate of the field effect transistor nanopore device; and
(d) measuring the resulting change in drain current across the source drain contacts.

2. The method of claim 1, wherein the FENT device comprises:
a silicon-on-insulator wafer substrate;
source and drain regions that are n+ doped;
a semiconductor channel that is continuous from source to drain regions and which narrows down into a conical point nanopore at the center;
a silicon gate that acts as a back/buried-gate;
a gate oxide layer that separates the silicon gate from the semiconductor channel; and
wherein the field effect transistor nanopore device is configured to operate in a fully depleted mode or partially depleted mode, such that a sensed chemical moiety and/or DNA base causes a measurable change in channel conductance.

3. The method of claim 1, wherein the chemical recognition molecule is placed on the semiconductor channel surface.

4. The method of claim 1, wherein the chemical recognition molecule is imidazole.

5. The method of claim 1, wherein the height of the chemical recognition molecule layer is within the range of from 3 Å to 200 Å.

6. The method of claim 1 wherein in the chemical recognition molecule comprises a unique molecule or a combination of molecules.

7. The method of claim 1, wherein the chemical recognition molecule is located at the edge of the nanopore.

8. The method of claim 1, wherein the chemical recognition molecule specifically interacts with the detected complex molecules.

9. The method of claim 1, wherein the chemical recognition molecule is an antibody coating.

10. The method of claim 1, wherein the chemical recognition molecules are complementary DNA bases.

11. The method of claim 1, wherein multiple layers of chemical or biomolecules are sequentially attached to the FENT device for detection of the complex molecules.

12. The method of claim 11, wherein sequentially attaching recognition molecules or chemical molecules or biomolecules comprises one or more of: chemical attachment, light directed attachment, electrochemical attachment, electrolysis-aided attachment, e-beam aided attachment, ion-beam aided attachment, and surface curvature aided attachment.

13. The method of claim 11, wherein different surface regions of the FENT device are coated with different chemical probes, biomolecules, or polymers.

14. The method of claim 1, wherein the FENT device is operated with silicon channel biased in one or more of inversion, accumulation, volume inversion, depletion, partial depletion, or full depletion.

15. The method of claim 1, wherein in the FENT device is biased with an AC signal to filter-out noise.

16. The method of claim 1, wherein the FENT device is used to count the material passing through the nanopore.

17. The method of claim 16, wherein each field effect transistor nanopore device within the array is made with different exterior coatings.

18. The method of claim 2, further comprising one or more electronic components configured so as to read out electrical signals, perform computational data analysis, and base identification.
